(19) United States
(12) Patent Application Publication
(10) Pub. No.: US 2005/0176631 A1
(43) Pub. Date: Aug. 11, 2005

(54) Method for reducing morbidity and mortality in critically ill patients

(76) Inventors: Josef Georg Heuer, Indianapolis, IN (US); Alexei Kharitonenkov, Carmel, IN (US)

Correspondence Address: ELI LILLY AND COMPANY;PATENT DIVISION, P.O. BOX 6288, INDIANAPOLIS, IN, 46206-6288, US

(21) Appl. No.: 10/499,608

(22) PCT Filed: Jan. 8, 2003
(86) PCT No.: PCT/US03/00010

Related U.S. Application Data
(60) Provisional application No. 60/348,890, filed on Jan. 15, 2002.

(51) Int. Cl. A61K 38/18

(52) U.S. Cl. 514/12

(57) ABSTRACT

This invention relates to a novel method of reducing the mortality and morbidity in critically ill patients which comprises administering to the patients an effective amount of FGF-21.

This invention relates to the use of fibroblast growth factor 21 (FGF-21) to reduce the morbidity and mortality associated with critically ill patients.

Critically ill patients requiring intensive care for an extended period of time have a high risk of death and substantial mortality. A common cause for admittance of patients to intensive care units (ICUs) is systemic inflammatory response syndrome (SIRS) associated with infectious insults (sepsis) as well as noninfectious pathologic causes such as pancreatitis, ischemia, multiple trauma and tissue injury, hemorrhagic shock, and immune-mediated organ injury.

A frequent complication of SIRS is the development of organ system dysfunction, including acute respiratory distress syndrome (ARDS), shock, renal failure, and multiple organ dysfunction syndrome (MODS), all of which amplify the risk of an adverse outcome. While many specialists believe that some type of nutritional support is beneficial to critically ill patients to help restore metabolic stability, the benefits and specifics of such support remain controversial due to the lack of well-controlled randomized clinical trials.

Because hyperglycemia and insulin resistance are common in critically ill patients given nutritional support, some ICUs administer insulin to treat excessive hyperglycemia in fed critically ill patients. In fact, recent studies document the use of exogenous insulin to maintain blood glucose at a level no higher than 110 mg per deciliter reduced morbidity and mortality among critically ill patients in the surgical intensive care unit, regardless of whether they had a history of diabetes (Van den Berghe, et al. N Engl J Med., 345(19):1359, 2001).

Fibroblast growth factors are large polypeptides widely expressed in developing and adult tissues (Baird et al., Cancer Cells, 3:239-243, 1991) and play crucial roles in multiple physiological functions. Fibroblast growth factor 21 (FGF-21) is a recently identified FGF which stimulates glucose uptake and enhances insulin sensitivity in 3T3-L1 adipocytes, an in vitro model utilized for the study of adipose tissue metabolism.

The present invention provides a more fundamental role for FGF-21 than merely indirectly regulating glucose levels in response to nutrient digestion. The present invention involves the discovery that FGF-21 affects the overall metabolic state and may counter-act negative side-effects that can occur during the body's stress response to sepsis as well as SIRS resulting from noninfectious pathologic causes. Thus, the present invention encompasses the use of FGF-21 to reduce the mortality and morbidity that occurs in critically ill patients.

The present invention encompasses a method for reducing mortality and morbidity associated with critically ill patients which comprises administering to the critically ill patients a therapeutically effective amount of FGF-21.

The present invention also encompasses a method of reducing mortality and morbidity in critically ill patients suffering from systemic inflammatory response syndrome (SIRS) associated with infectious insults as well as noninfectious pathologic causes which comprises administering to the critically ill patients a therapeutically effective amount of FGF-21. Examples of conditions that involve SIRS include sepsis, pancreatitis, ischemia, multiple trauma and tissue injury, hemorrhagic shock, immune-mediated organ injury, acute respiratory distress syndrome (ARDS), shock, renal failure, and multiple organ dysfunction syndrome (MODS).

The present invention also encompasses a method of reducing mortality and morbidity in critically ill patients suffering from respiratory distress.

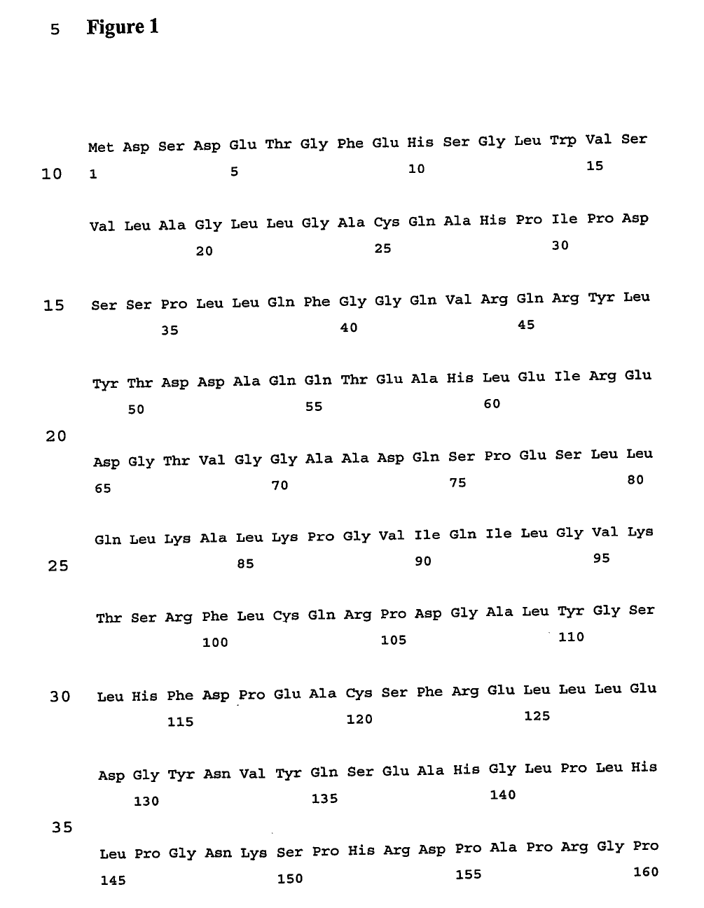

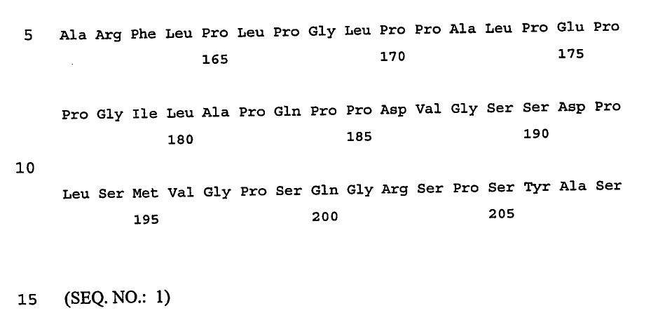

FIG. 1 shows the 208 amino acid sequence of fibroblast growth factor 21 (SEQ. NO: 1).

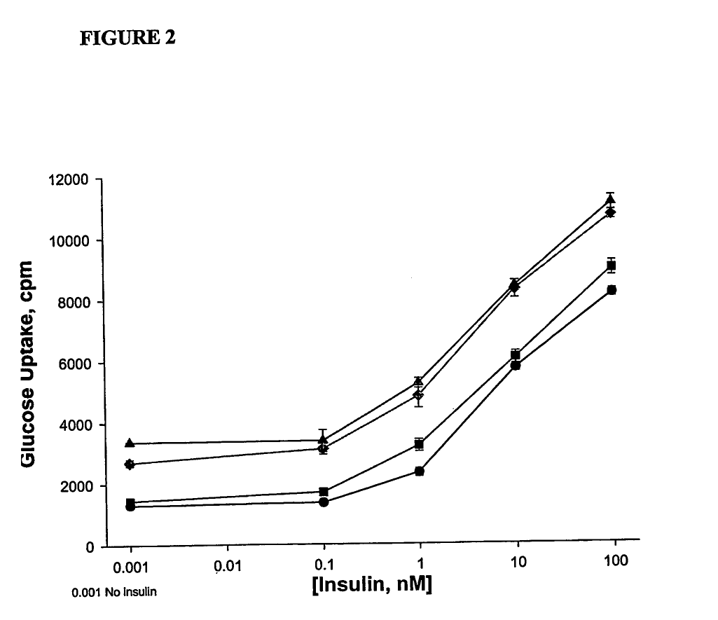

FIG. 2 shows FGF-21 stimulation of glucose uptake in 3T3-L1 adipocytes upon acute or chronic pretreatment in the presence of insulin. ● Control; ▪ FGF-21 (1 μg/ml), acute pretreatment (20 minutes); ▴ FGF-21 (1 μg/ml), chronic pretreatment (72 hours); ♦ FGF-21 (1 μg/ml), chronic pretreatment (72 hours)+acute pretreatment (20 minutes).

Methods and compositions, in particular medicaments (pharmaceutical compositions or formulations) using FGF-21 are effective in reducing the mortality and morbidity for critically ill patients. In addition, such compositions are effective in reducing the mortality and morbidity associated with systemic inflammatory response syndrome. Moreover, such compositions are effective in reducing the mortality and morbidity associated with the stress response that occurs as a result of certain traumas or conditions that often lead to various degrees of respiratory distress. For the purposes of the present invention a “subject” or “patient” is preferably a human, but can also be an animal, e.g., companion animal (e.g., dogs, cats, and the like), farm animals (e.g., cows, sheep, pigs, horses, and the like) and laboratory animals (e.g., rats, mice, guinea pigs, and the like).

The practice of critical care medicine is hospital-based and is dedicated to and defined by the needs of the critically ill patients. Critically ill patients include those patients who are physiologically unstable requiring continuous, coordinated physician, nursing, and respiratory care. This type of care necessitates paying particular attention to detail in order to provide constant surveillance and titration of therapy. Critically ill patients include those patients who are at risk for physiological decompensation and thus require constant monitoring such that the intensive care team can provide immediate intervention to prevent adverse occurrences. Critically ill patients have special needs for monitoring and life support which must be provided by a team that can provide continuous titrated care.

The present invention encompasses a method of reducing the mortality and morbidity in these critically ill patients through the administration of FGF-21. The critically ill patients encompassed by the present invention generally experience an unstable hypermetabolic state. This unstable metabolic state is due to changes in substrate metabolism which may lead to relative deficiencies in some nutrients. Generally there is increased oxidation of both fat and muscle.

The critically ill patients wherein the administration of FGF-21 can reduce the risk of mortality and morbidity are preferably patients that experience systemic inflammatory response syndrome or respiratory distress. A reduction in morbidity means reducing the likelihood that a critically ill patient will develop additional illnesses, conditions, or symptoms or reducing the severity of additional illnesses, conditions, or symptoms. For example reducing morbidity may correspond to a decrease in the incidence of bacteremia or sepsis or complications associated with multiple organ failure.

“Systemic inflammatory response syndrome (SIRS)” as used herein describes an inflammatory process associated with a large number of clinical conditions and includes, but is not limited to, more than one of the following clinical manifestations: (1) a body temperature greater than 38° C. or less than 36° C.; (2) a heart rate greater than 90 beats per minute; (3) tachypnea, manifested by a respiratory rate greater than 20 breaths per minute, or hyperventilation, as indicated by a PaCo₂ of less than 32 mm Hg; and (4) an alteration in the white blood cell count, such as a count greater than 12,000/cu mm, a count less than 4,000/cu mm, or the presence of more than 10% immature neutrophils. These physiologic changes should represent an acute alteration from baseline in the absence of other known causes for such abnormalities, such as chemotherapy, induced neutropenia, and leukopenia.

“Sepsis” as used herein is defined as a SIRS arising from infection. Noninfectious pathogenic causes of SIRS may include pancreatitis, ischemia, multiple trauma and tissue injury i.e. crushing injuries or severe burns, hemorrhagic shock, immune-mediated organ injury, and the exogenous administration of such putative mediators of the inflammatory process as tumor necrosis factor and other cytokines.

Septic shock and multi-organ dysfunction are major contributors to morbidity and mortality in the ICU setting. Sepsis is associated with and mediated by the activation of a number of host defense mechanisms including the cytokine network, leukocytes, and the complement cascade, and coagulation/fibrinolysis systems including the endothelium. Disseminated intravascular coagulation (DIC) and other degrees of consumption coagulopathy associated with fibrin deposition within the microvasculature of various organs, are manifestations of sepsis/septic shock. The downstream effects of the host defense response on target organs is an important mediator in the development of the multiple organ dysfunction syndrome (MODS) and contributes to the poor prognosis of patients with sepsis, severe sepsis and sepsis complicated by shock.

“Respiratory distress” as used herein denotes a condition wherein patients have difficulty breathing due to some type of pulmonary dysfunction. Often these patients exhibit varying degrees of hypoxemia that may or may not be refractory to treatment with supplemental oxygen.

Respiratory distress may occur in patients with impaired pulmonary function due to direct lung injury or may occur due to indirect lung injury such as in the setting of a systemic process. In addition, the presence of multiple predisposing disorders substantially increases the risk, as does the presence of secondary factors such as chronic alcohol abuse, chronic lung disease, and a low serum pH.

Some causes of direct lung injury include pneumonia, aspiration of gastric contents, pulmonary contusion, fat emboli, near-drowning, inhalation injury, high altitude and reperfusion pulmonary edema after lung transplantation or pulmonary embolectomy. Some causes of indirect lung injury include sepsis, severe trauma with shock and multiple transfusions; cardiopulmonary bypass, drug overdose, acute pancreatitis, and transfusions of blood products.

One class of pulmonary disorders that causes respiratory distress are associated with the syndrome known as Cor Pulmonale. These disorders are associated with chronic hypoxemia resulting in raised pressure within the pulmonary circulation called pulmonary hypertension. The ensuing pulmonary hypertension increases the work load of the right ventricle, thus leading to its enlargement or hypertrophy. Cor Pulmonale generally presents as right heart failure defined by a sustained increase in right ventricular pressures and clinical evidence of reduced venous return to the right heart.

Chronic obstructive pulmonary diseases (COPDs) which include emphysema and chronic bronchitis also cause respiratory distress and are characterized by obstruction to air flow. COPDs are the fourth leading cause of death and claim over 100,000 lives annually.

Acute respiratory distress syndrome (ARDS) is generally progressive and characterized by distinct stages. The syndrome is generally manifested by the rapid onset of respiratory failure in a patient with a risk factor for the condition. Arterial hypoxemia that is refractory to treatment with supplemental oxygen is a characteristic feature. There may be alveolar filling, consolidation, and atelectasis occurring in dependent lung zones; however, non-dependent areas may have substantial inflammation. The syndrome may progress to fibrosing alveolitis with persistent hypoxemia, increased alveolar dead space, and a further decrease in pulmonary compliance. Pulmonary hypertension which results from damage to the pulmonary capillary bed may also develop.

The severity of clinical lung injury varies. Both patients with less severe hypoxemia as defined by a ratio of the partial pressure of arterial oxygen to the fraction of inspired oxygen as 300 or less and patients with more severe hypoxemia as defined by a ratio of 200 or less are encompassed by the present invention. Generally, patients with a ratio 300 or less are classified as having acute lung injury and patients with having a ratio of 200 or less are classified as having acute respiratory distress syndrome.

The acute phase of acute lung injury is characterized by an influx of protein-rich edema fluid into the air spaces as a consequence of increased vascular permeability of the alveolar-capillary barrier. The loss of epithelial integrity wherein permeability is altered can cause alveolar flooding, disrupt normal fluid transport which affects the removal of edema fluid from the alveolar space, reduce the production and turnover of surfactant, lead to septic shock in patients with bacterial pneumonia, and cause fibrosis. Sepsis is associated with the highest risk of progression to acute lung injury.

In conditions such as sepsis, where hypermetabolism occurs, there is an accelerated protein breakdown both to sustain gluconeogenesis and to liberate the amino acids required for increased protein synthesis. Hyperglycemia may be present and high concentrations of triglycerides and other lipids in serum may be present.

For patients with compromised respiratory function, hypermetabolism may affect the ratio of carbon dioxide production to oxygen consumption. This is known as the respiratory quotient (R/Q) and in normal individuals is between about 0.85 and about 0.90. Excess fat metabolism has a tendency to lower the R/Q whereas excess glucose metabolism raises the R/Q. Patients with respiratory distress often have difficulty eliminating carbon dioxide and thus have abnormally high respiratory quotients.

The critically ill patients encompassed by the present invention also generally experience a particular stress response characterized by a transient down-regulation of most cellular products and the up-regulation of heat shock proteins. Furthermore, this stress response involves the activation of hormones such as glucagon, growth hormone, cortisol, and pro- and anti- inflammatory cytokines. While this stress response appears to have a protective function, the response creates additional metabolic instability in these critically ill patients. For example, activation of these specific hormones causes elevations in serum glucose which results in hyperglycemia. In addition, damage to the heart and other organs may be exacerbated by adrenergic stimuli. Further, there may be changes to the thyroid which may have significant effects on metabolic activity.

Fibroblast growth factors are large polypeptides widely expressed in developing and adult tissues (Baird et al., Cancer Cells, 3:239-243, 1991) and play crucial roles in multiple physiological functions. Fibroblast growth factor 21 (FGF-21) is a recently identified FGF which has been reported to be preferentially expressed in the liver (Nishimura et al., Biochimica et Biophysica Acta, 1492:203-206, 2000; WO01/36640; and WO01/18172) and described as a treatment for ischemic vascular disease, wound healing, and diseases associated with loss of pulmonary, bronchia or alvelor cells or function and numerous other disorders.

We have discovered that FGF-21 significantly improved the survival of ob/ob mice in an in vivo septic shock model, Example 3. Furthermore, we have also discovered that FGF-21 stimulates glucose uptake and enhances insulin sensitivity in 3T3-L1 adipocytes, an in vitro model utilized for the study of adipose tissue metabolism, Example 1. FGF-21 is shown to stimulate glucose uptake in 3T3-L1 adipocytes in a concentration dependent manner at a sub-optimal concentration of insulin (5nM), Example 2, Table 1. In FIG. 2, FGF-21 is shown to positively influence insulin-dependent glucose uptake in 3T3-L1 adipocytes upon 72 hour treatment.

FGF-21 is uniquely suited to help restore metabolic stability in metabolically unstable critically ill patients. FGF-21 is unique in that it stimulates glucose uptake and enhances insulin sensitivity. Further, FGF-21 has a wide biological role in man, affecting organs through mechanisms that may not necessarily be related to glycemia. Thus, FGF-21 is ideally suited to treat critically ill patients.

The FGF-21 useful in the methods of the present invention includes human FGF-21 (the amino acid sequence of which is as shown in SEQ ID NO:1), FGF-21 analogs, FGF-21 derivatives, and other agonists of the FGF-21 receptor, hereinafter collectively known as FGF-21 compounds. FGF-21 analogs have sufficient homology to FGF-21 such that the compound has the ability to bind to the FGF-21 receptor and initiate a signal transduction pathway resulting in glucose uptake stimulation or other physiological effects as described herein. For example, FGF-21 compounds can be tested for glucose uptake activity using a cell-based assay such as that described in Example 2.

To determine whether an FGF-21 compound is suitable for the methods encompassed by the present invention an in vivo survival study can be conducted as described in Example 3.

A FGF-21 compound also includes a “FGF-21 derivative” which is defined as a molecule having the amino acid sequence of FGF-21 or of a FGF-21 analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The α-carbon of an amino acid may be mono- or dimethylated.

The FGF-21 administered according to this invention may be generated and/or isolated by any means known in the art such as described in Sambrook et al., Molecular Cloning: A Laboratory Manual, Cold Spring Harbor Laboratory Press, NY (1989).

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for FGF-21.

Compositions

FGF-21 of the present invention may be formulated as a pharmaceutically acceptable compositions. A pharmaceutically acceptable drug product may have the FGF-21 compound combined with a pharmaceutically-acceptable buffer, wherein the pH is suitable for parenteral administration and adjusted to provide acceptable stability and solubility properties. Pharmaceutically-acceptable anti-microbial agents may also be added. Meta-cresol and phenol are preferred pharmaceutically-acceptable anti-microbial agents. One or more pharmaceutically-acceptable salts may also be added to adjust the ionic strength or tonicity. One or more excipients may be added to further adjust the isotonicity of the formulation. Glycerin is an example of an isotonicity-adjusting excipient.

“Pharmaceutically acceptable” means suitable for administration to a human. A pharmaceutically acceptable formulation does not contain toxic elements, undesirable contaminants or the like, and does not interfere with the activity of the active compounds therein.

Pharmaceutically acceptable compositions comprised of a FGF-21 compound may be administered by a variety of routes such as orally, by nasal administration, by inhalation, or parenterally. Parenteral administration can include, for example, systemic administration, such as by intramuscular, intravenous, subcutaneous, or intraperitoneal injection. Because the present invention is primarily applicable to a method of treating critically ill patients who have been admitted to a hospital ICU, intravenous administration is preferred. Intravenous administration may use continuous infusion or a bolus injection. Continuous infusion means continuing substantially uninterrupted the introduction of a solution into a vein for a specified period of time. A bolus injection is the injection of a drug in a defined quantity (called a bolus) over a period of time.

If subcutaneous administration is used or an alternative type of administration, the FGF-21 compounds should be derivatized or formulated such that they have a protracted profile of action.

A “therapeutically effective amount” of a FGF-21 compound is the quantity which results in a desired effect without causing unacceptable side-effects when administered to a subject. A desired effect can include an amelioration of symptoms associated with the disease or condition, a delay in the onset of symptoms associated with the disease or condition, and increased longevity compared with the absence of treatment. In particular, the desired effect is a reduction in the mortality and morbidity associated with critical illnesses.

To achieve efficacy while minimizing side effects, the plasma levels of a FGF-21 compound should not fluctuate significantly once steady state levels are obtained during the course of treatment. Levels do not fluctuate significantly if they are maintained within the ranges described herein once steady state levels are achieved throughout a course of treatment. Those skilled in the art can readily optimize pharmaceutically effective dosages and administration regimens for therapeutic compositions comprising FGF-21, as determined by good medical practice and the clinical condition of the individual patient. Generally, the formulations are constructed so as to achieve a constant local concentration of about 100 times the serum level of the growth factor or 10 times the tissue concentration, as described in Buckley et al (Proc Natl Acad Sci (USA) 82:7340-7344, 1985). Based on an FGF concentration in tissue of 5-50 ng/g wet weight, release of 50-5000 ng FGF-21 per hour is acceptable. Preferably, release of 50-4000; 50-3000; 50-2000; 50-1000; 50-500; 50-250; or 50-100 ng of FGF-21 per hour is acceptable. The appropriate dose of FGF-21 administered will result in a reduction in the mortality and morbidity associated with critical illnesses.

FGF-21 compounds can be used in combination with a variety of other medications that are routinely administered to critically-ill patients admitted to a hospital ICU. For example, these critically ill patients may be given prophylaxis for deep venous thrombosis or pulmonary emboli which consists of heparin (usually 5,000 units q 12 hours), lovenox or an equivalent thereof. Low-doses of coumadin may be used as an anticoagulant. Often ICU patients receive an H2 blocker, an antacid, omeprazole, sucraflate or other drugs to counter-act potential gastroduodenal ulceration and bleeding. Antibiotics are commonly given to patients in the ICU. Patients with sepsis or multisystem organ failure may be given Nystatin or Fluconazole for candidal prophylaxis.

In another aspect of the present invention, FGF-21 for use as a medicament for the treatment of critically ill patients is contemplated.

Having generally described the invention, the same will be more readily understood by reference to the following examples, which are provided by way of illustration and are not intended as limiting.

### EXAMPLE 1

### Tissue Distribution of FGF-21-Encoding mRNA

Northern blot analysis is carried out to examine expression of FGF-21 encoding mRNA in human tissues, using methods described by, among others, Sambrook, et al., cited above. A cDNA probe preferably encoding the entire FGF-21 polypeptide is labeled with ³²P using the Rediprime™ DNA labeling system (Amersham Life Science), according to the manufacturer's instructions. After labeling, the probe is purified using a CHROMA SPIN-100™ column (Clontech Laboratories, Inc.), according to the manufacturer's protocol number PT1200-1. The purified and labeled probe is used to examine various human tissues for FGF-21 mRNA.

Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at −70° C. overnight, and developed according to standard procedures.

The above technique demonstrates that FGF-21 is expressed primarily in the liver, kidney and muscle.

### EXAMPLE 2

### Glucose Uptake in 3T3-1 Adipocytes

3T3-L1 cells are obtained from the American Type Culture Collection (ATCC, Rockville, Md.). Cells are cultured in growth medium (GM) containing 10% iron-enriched fetal bovine serum in Dulbecco's modified Eagle's medium. For standard adipocyte differentiation, 2 days after cells reached confluency (referred as day 0), cells are exposed to differentiation medium (DM) containing 10% fetal bovine serum, 10 μg/ml of insulin, 1 μM dexamethasone, and 0.5 μM isobutylmethylxanthine, for 48 h. Cells then are maintained in post differentiation medium containing 10% fetal bovine serum, and 10 μg/ml of insulin.

Glucose Transport Assay—Hexose uptake, as assayed by the accumulation of 0.1 mM 2-deoxy-D-[¹⁴C]glucose, is measured as follows: 3T3-L1 adipocytes in 12-well plates are washed twice with KRP buffer (136 mM NaCl, 4.7 mM KCl 10 M NaPO₄, 0.9 mM CaCl₂, 0.9 mM MgSO₄, pH 7.4) warmed to 37° C. and containing 0.2% BSA, incubated in Leibovitz's L-15 medium containing 0.2% BSA for 2 h at 37° C. in room air, washed twice again with KRP containing, 0.2% BSA buffer, and incubated in KRP, 0.2% BSA buffer in the absence (Me₂SO only) or presence of wortmannin for 30 min at 37° C. in room air. Insulin is then added to a final concentration of 100 nM for 15 min, and the uptake of 2-deoxy-D-[¹⁴C]glucose is measured for the last 4 min. Nonspecific uptake, measured in the presence of 10 μM cytochalasin B, is subtracted from all values. Protein concentrations are determined with the Pierce bicinchoninic acid assay. Uptake is measured routinely in triplicate or quadruplicate for each experiment. FGF-21 stimulation of glucose uptake in 3T3-L1 adipocytes in a concentration dependent manner, performed at a sub-optimal concentration of insulin (5 nM) is shown in Table 1. The effect of acute and chronic pretreatment of 3T3-L1 adipocytes with FGF-21 in the presence of insulin is shown in FIG. 2, indicating that FGF-21 positively influences insulin-dependent glucose uptake upon 72 hour treatment.

TABLE 1

| | Glucose Uptake |
|---|---|
| FGF-21 (μg/ml) | (CPM) |
| 0 | 7200 |
| 0.01 | 7650 |
| 0.1 | 7850 |
| 1.0 | 8200 |
| 10.0 | 8400 |

### EXAMPLE 3

### In Vivo Model of Sepsis

An in vivo model of sepsis is used to study the effect of FGF-21 on animal survival. Female ob/ob mice (8-9 weeks) are challenged with an i.p. injection of lipopolysaccharide (LPS) (27.5 ug LPS/g mouse in 100 ul PBS). FGF-21 or human serum albumin (50 ug per injection) are injected BID by s.c. injection in 200 ul of PBS beginning at 1 hour post LPS and continuing for 48 hours. The mice are monitored 3 times daily for survival over a 54 hour time period.

A summary of four separate experiments indicates that after 54 hours, 95% of the mice treated with human serum albumin died while 58% of the mice treated with FGF-21 survived (p-value=0.05). Furthermore, after seven days (168 hours), 100% of the mice treated with human serum albumin died while 20% of the mice treated with FGF-21 still survived.

### EXAMPLE 4

### Transcriptional Profiling of FGF-21 Treated 3T3-L1 Adipocytes

3T3-L1 adipocytes are treated with FGF-21 and then harvested, homogenized and the RNA is extracted. Briefly, cell samples were homogenized in 1 ml TRIzol reagent (GibcoBRL) per 50 mg of tissue using a power homogenizer. RNA was extracted using TRIzol reagent according to manufacturer's instructions.

RNA is prepared for GeneChip hybridization on the Human FL arrays (Affymetrix). After hybridization and scanning, the genes are rank ordered according to the Average Difference Intensity (ADI) between the control and the FGF-21 treated samples using a statistical comparison analysis.

To confirm the validity of these changes, the expression of several of the genes from the 3T3-L1 adipocytes are examined using a semi-quantitative RT-PCR assay. The same mRNA pools are used for both the microarrays and the RT-PCR assays. Genes upregulated by FGF-21 treatment of 3T3-L1 adipocytes are GADD45 and chop-10, both of which are normally upregulated during nutritional stress.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A method of reducing the mortality and morbidity in critically ill patients which comprises administering to the patients an effective amount of FGF-21.

13. The method of claim 12 wherein said critically ill patients are suffering from systemic inflammatory response syndrome.

14. The method of claim 12 wherein said critically ill patients are suffering from respiratory distress.

15. The method of claim 12 wherein the patients have acute lung injury.

16. The method of claim 12 wherein the patients have acute respiratory distress syndrome.

17. The method of claim 12 wherein the patients have multiple organ dysfunction syndrome.

18. The method of claim 12 wherein the patients have sepsis.

19. The method of claim 12 wherein FGF-21 is administered via continuous infusion.

20. The method of claim 12 wherein FGF-21 is administered via a bolus injection.


SEQUENCE LISTING (1 sequence)

SEQ ID NO: 1 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS